(19) United States
(12) Patent Application Publication
(10) Pub. No.: US 2007/0014858 A1
(43) Pub. Date: Jan. 18, 2007

(54) Method for controlling lag time of in-situ passageway formation in osmotic delivery system

(75) Inventors: Jen-Sen Wu, Taipei (TW); Yih-Yih Lin, Taipei (TW); Shou-Chiung Chen, Taipei (TW); Yi-Pin Huang, Taipei (TW); Hui-Ju Chan, Taipei (TW); Pei-Chun Kuo, Taipei (TW); Chun-Yu Lin, Taipei (TW)

Correspondence Address: FISH & RICHARDSON PC, P.O. BOX 1022, MINNEAPOLIS, MN, 55440-1022, US

(73) Assignee: EMPAX PHARMA INC.

(21) Appl. No.: 11/481,749

(22) Filed: Jul. 6, 2006

(30) Foreign Application Priority Data
Jul. 12, 2005 (TW) 94123473

(51) Int. Cl. A61K 9/46; A61K 9/24

(52) U.S. Cl. 424/466; 424/473

(57) ABSTRACT

An osmotically controlled delivery system includes a solid core having a shallow indentation on a surface of the core, and a semipermeable membrane enclosing the solid core. The solid core is made by a drug composition being caopable of generating an osmotically effective pressure, and the semipermeable membrane is relatively thinner at the shallow indentation. An in-situ exit passageway is formed in the indentation position when external aqueous fluids are imbibed through the semipermeable membrane into the dosage form by an osmotic pressure gradient. A process for forming an in-situ exit passageway of an osmotic delivery dosage form, a controlled onset dosage form, and a method for controlling the lag time of in-situ passageway formation are also disclosed.

### RELATED APPLICATIONS

The present application is based on, and claims priority from, Taiwan Application Serial Number 94123473, filed Jul. 12, 2005, the disclosure of which is hereby incorporated by reference herein in its entirety.

### BACKGROUND OF THE INVENTION

1. Field of the Invention

The present invention relates to a dosage form of controlled onset and drug release and a method thereof. More specifically, the present invention relates to an osmotic delivery dosage form and a method for controlling the lag time of in-situ passageway formation.

2. Description of Related Art

Recently, in addition to the drug discovery, the control of drug release rates in the body is an important task to the pharmaceutical industry. By maintaining the drug concentration in the blood, this controlled release dosage form can reduce the side effect, extend the reaction time, reduce the frequency of drug administration, increase the compliance of patients, and further increase the efficacy of the treatments of chronic diseases.

Among the controlled release dosage forms, the oral administration is the most convenient one. According to the release mechanism, they can be categorized into three systems: matrix, reservoir and osmotic pump delivery system. The osmotic pump system can deliver drugs at a zero-order release rate to maintain the drug concentration in the blood. Furthermore, the release rate of the osmotic pump is not affected by factors in the gastrointestinal tract (for example, pH value, food or the gastrointestinal movement). Therefore, it is more efficient than the other two systems. The osmotic pump uses a semipermeable membrane to cover a drug and osmotic substances, allowing only water to permeate. When external aqueous fluids are imbibed through the semipermeable membrane into the core by an osmotic pressure gradient, the drug is released from a hole in the membrane.

In 1974, U.S. Pat. No. 3,845,770 first disclosed an osmotic delivery system which was made with a semipermeable membrane surrounding a compartment containing agent and having a passageway for delivering the agent from compartment to the external fluid. The semipermeable membrane is permeable to an external fluid and substantially impermeable to the agent. The agent in the compartment was soluble to the imbibed external fluids and exhibits an osmotic pressure gradient across the membrane against the fluid. External fluids were imbibed through the semipermeable membrane into the compartment at a rate determined by the permeability of the membrane and the osmotic pressure gradient. The drug was dissolved in the imbibed fluids and released through the passageway at a controlled rate. A disadvantage of this dosage form was that a passageway needed to be formed after the drug was covered by the membrane. Numbers of patents disclosed methods to improve the preparation of the passageway. At present, the passageway of the osmotic pump controlled release system is generally prepared by laser drilling. However, the cost is high and the process is complicated.

In 1976, U.S. Pat. No. 3,952,741 disclosed an osmotic dispenser wherein weak spots on the membrane were pressed to open by a pressure gradient due to water absorption and the drugs were released. By controlling the time at which the rupture of weak spots take place within the dosage form, a pulsatile delivery of the drug was possible. A disadvantage of this dosage form was that the design could only be used in pulsatile delivery in which a certain amount of drug was released interseptally. In order to reach a consistent release rate, the dosage form had to comprise many units with short release intervals, which was difficult in practice.

In 1977, U.S. Pat. No. 4,016,880 disclosed a method wherein a passageway in the membrane was formed when external aqueous fluids were imbibed through the membrane into the dosage form by an osmotic pressure gradient. With the mechanism of the osmotic pump, the drug was released consistently. A disadvantage of this dosage form was that the coating membrane comprised materials weakening the membrane in order to form the passageway. Therefore the toughness and flexibility of the membrane were lower. In clinical use, the membrane might be broken and result in drug dumping.

In 1978, U.S. Pat. No. 4,088,864 disclosed a method wherein a laser drilling technique was applied to produce the passageway. The size of the passageway was regulated by controlling the laser energy and exposing time to the laser. A machine which was able to mass-produce the osmotic pump tablet by laser drilling was also disclosed. The disadvantages of this design include expensive equipment and high production cost. Moreover, the production rate was limited by the cycle time of the laser beam. At present, the passageway of the osmotic pump controlled release system is generally prepared by laser drilling. However, the cost is high and the process is complicated.

In 1981, U.S. Pat. No. 4,271,113 disclosed a method wherein a deep hole was formed by a special mold filling process during or after a tablet being compressed. The deep hole was not covered completely by a semipermeable membrane, therefore a passageway was formed. A disadvantage of this method was: the hole on the surface of the tablet was deep to prevent the membrane from covering the hole in the following membrane preparing process. The protrusion on the concavity side of the “mold” therefore was required to be accordingly long. It was liable to damage during usage. The repeatability was questionable in mass production.

In 1986, U.S. Pat. No. 4,612,008 disclosed a push-pull osmotic pump. A drug layer was adjoined to a passageway. Under the drug layer was a push layer containing an osmopolymer. When the push layer absorbed water, it swelled constantly and pushed the drug to be released from the passageway. When the solubility of the drug was too high or too low for the osmotic pump delivery system, the controlled releasing rate of the drug could reach zero order by using the push-pull osmotic pump. The disadvantage of this pump was that laser drilling technique was required and the cost was high and the production was slow. Besides, the laser drilling technique had to be controlled to drill only on the surface of the membrane of the drug layer. The manufacturing process was complicated.

In 1990, U.S. Pat. No. 4,968,507 disclosed a method wherein the semipermeable membrane contained water soluble substances. A porous semipermeable membrane was formed when the water soluble substances were dissolved. The disadvantage was: a) when low molecular weight crystalline compounds, such as sugars, were used as the water soluble substances, crystallization on the membrane was seen after storage for a while. b) The release rate of the drug was determined by both mechanisms of osmotic pump and diffusion. The release of the drug may be affected by the pH values in the gastrointestinal tract.

In 1991, U.S. Pat. No. 5,071,607 disclosed a special ingot mold, with which tablets were coated by semipermeable membranes. During the compression coating process, a passageway was formed simultaneously. The disadvantage of this method was that the design of the mold was complicated.

In 1998, U.S. Pat. No. 5,736,159 disclosed a method, wherein polymers which swell as absorbed water were added to the tablets. Holes (passageway) were formed on the periphery of the tablets by the pressure from the swelling action. The disadvantages were: a) a thin membrane is more likely to be affected by factors in the environment. The variability of drug release might cause problems in the gastrointestinal tract. b) The size and shape of the holes were variable.

In summary, the prior techniques for improving the preparation of the drug passageway include: mechanical drilling to form the passageway, laser drilling in the membrane (U.S. Pat. No. 4,088,864), forming a deep hole on the surface of the tablet so as to make the membrane cover incompletely (U.S. Pat. No. 4,271,113), and compressing to cover the semipermeable membrane on the tablets and meanwhile preparing the passageway (U.S. Pat. No. 5,071,607). However, special machines were required in these methods, and there are disadvantages, such as high costs and low production. On the other hand, the passageway can be formed after entering the human body. In these methods the composition of the semipermeable membrane has to be changed, or the coating level has to be limited. Therefore the applications of these osmotic pump systems were limited. For example, when the semipermeable membrane comprises water soluble substances, a porous semipermeable membrane is formed in the human body while the water soluble substances are dissolved. The releasing rate of the drug is determined by both, osmotic pump and diffusion. The dissolving rate might be affected by the pH value in the gastrointestinal tract (U.S. Pat. No. 4,968,507). The other method in U.S. Pat. No. 4,016,880 discloses forming a passageway in the membrane when external aqueous fluids were imbibed through the membrane into the dosage form by an osmotic pressure gradient. Since the coating membrane comprised materials weakening the membrane in order to form the passageway, the membrane might be broken by gastrointestinal movements in the human body, leading to the risks of releasing a great quantity of drugs. In U.S. Pat. No. 5,736,159, passageways were formed on the periphery of the tablets by the internal pressure. However, the coating level of this membrane was accordingly low for the application of lower internal pressure, since higher internal pressure can enlarge the passageway to cracks in the membrane. Therefore, these thin membranes are likely to be affected by factors in the environment and the variability of drug release might cause problems in the gastrointestinal tract.

### SUMMARY OF THE INVENTION

Accordingly, one object of the present invention is to provide a method for forming an in-situ passageway of an osmotic delivery dosage form. This passageway is formed at an anticipant position, and the release rate of the drug is quasi-zero order. It provides a simple and convenient way for a controlled release drug and the cost is lower than the above-noted prior art. In addition, the present invention also discloses a controlled onset dosage form, and a method for controlling the lag time of in-situ passageway formation, since certain patients need to take medication at certain times.

The present invention provides an osmotic delivery dosage form, which comprises: a) a solid core having a shallow indentation on the surface of the core, which contains active drugs and pharmaceutically acceptable salts, and b) a semipermeable membrane having a substantially intact surface enclosing the solid core, wherein the semipermeable membrane completely covers the shallow indentation and is relatively thinner at an indentation position. When external aqueous fluids are imbibed through the semipermeable membrane into the dosage form by an osmotic pressure gradient, an in-situ exit passageway is formed in the indentation position. The drug is subsequently released through the passageway. The dosage form is preferably a tablet.

In the present invention, a shallow indentation is produced on the surface of the core of a dosage form or a tablet. When the dosage form is completely covered with the semipermeable membrane, a relatively thinner membrane is formed around the indentation surface. The depth of the shallow indentation is about 100-300 μm, preferably about 150-250 μm. When the dosage form or tablet of the present invention is completely covered with the semipermeable membrane, instead of the periphery of the dosage form or tablet, the shallow indentation becomes the weakest point of the dosage form due to a relatively thinner membrane is formed around the indentation surface. Since the semipermeable membrane can allow only external aqueous fluids to permeate but not the active compounds, external aqueous fluids (for example, water, gastric and intestinal juice, or imitative gastrointestinal juice) are imbibed through the semipermeable membrane into the dosage form by an osmotic pressure gradient, and an internal pressure is formed to press the thinner membrane to open. An in-situ exit passageway is formed in the indentation position. The size and position of the passage way is limited to the shallow indentation. It will not widen to become a big crack. Therefore, the depth of the membrane of the dosage form in the present invention is not limited. It is preferably about 100-400 μm.

The materials used for the semipermeable membrane in the invention are well-known in the pharmaceutical industry. For example, the commercially available non-plasticised cellulose acetate, plasticised cellulose triacetate, agar acetate, pentacglucose acetate, dextran acetate, cellulose acetate methylurethane, cellulose acetate phthalate, cellulose acetate ethylurethane, cellulose acetate succinate, cellulose acetate dimethylglycine, cellulose acetate ethanecarbonate, cellulose acetate methanesulfonate, cellulose acetate butanesulfonate, cellulose acetate propionate, vinyl methyl ether polymer, cellulose acetate coctanoate, cellulose acetate laurate, cellulose acetate p-toluenesulfonate, ethyl cellulose, locust bean gum triacetate, cellulose acetate with acetyl hydroxyethylcellulose, hydroxation ethylene vinyl acetate, membrane material made with expoxy polymer, alkylidene oxide-alkyl glycidyl ether, polyurethane, polyglycolic acid, and the well-known polyoxygen-polyanionic membrane.

The technique to prepare the shallow indentation includes using a mold with a prominence on a surface to compress or mold tablets. The height of the prominence accords with the design of the mold for usual logos or names. Therefore the ordinary compressing equipment is also suitable for the present invention. Compared to the method disclosed in U.S. Pat. No. 4,271,113, wherein a deep hole on the surface is required so as to not be covered by a membrane completely, the mold for the present invention is simpler.

Apart from the traditional tablet compressing equipment, a standard concave mold with prominence can be used to prepare the shallow indentation. The shape of the prominence can be anything adaptable, for example, a round shape, a square, a rhombus, any other adaptable shapes or a mixture of the above (see FIG. 1). FIG. 1a shows a 3-dimensional view of a mold. FIG. 1b shows a cross-section view of a mold. FIG. 1c shows a top view of a mold with a round prominence. FIG. 1d shows a top view of a mold with a square prominence. FIG. 1e shows a top view of a mold with a rhombic prominence.

Depending on the needs, the solid core of the dosage form in the present invention can also comprise: an effervescent substance, an osmagent, an osmopolymer or a mixture of the above. In one embodiment of the present invention, the solid core is a double-layer tablet. The upper layer comprises a drug, and the lower layer is a push layer containing an osmopolymer. The shallow indentation is on the surface of the drug containing layer. In another embodiment of the present invention, the solid core is a triple-layer tablet. The upper layer is an effervescent layer comprising an effervescent substance; the middle layer comprises a drug; and the lower layer is a push layer containing an osmopolymer. The shallow indentation is on the surface of an effervescent layer. Hence the dosage form of the present invention includes single-layer tablets, double-layer tablets, triple-layer tablets, compression-coated tablets or multi-layer tablets.

The present invention also provides an osmotic delivery dosage form, which comprises: a) a solid core having a shallow indentation on a surface of the core, which contains a drug containing layer with an active ingredient and pharmaceutically acceptable salts thereof, and a push layer comprising an osmopolymer; and b) a semipermeable membrane having a substantially intact surface enclosing the solid core, wherein the semipermeable membrane completely covers the shallow indentation and is relatively thinner at an indentation position, whereby an in-situ exit passageway is formed in the indentation position when external aqueous fluids are imbibed through the semipermeable membrane into the dosage form by an osmotic pressure gradient. The drug is subsequently released through the passageway.

Preferably, the present invention provides a double-layer dosage form 10 comprising a drug containing layer 2 with a shallow indentation 1 on the surface, a push layer 3, and a semipermeable membrane 8. The construction is shown in FIG. 2. FIG. 2a is a 3-dimensinal view of a double-layer dosage form. FIG. 2b shows a cross-section view of a double-layer dosage form. In addition, one embodiment of the osmotic delivery dosage form in the present invention is a triple-layer tablet. The construction is shown in FIG. 3. A push layer 3 is in the middle. The upper and lower layers are both drug containing layers 2 with a shallow indentation 1 on the surface. The drugs on these two layers can be either identical or different. The triple-layer tablet is completely covered with the semipermeable membrane 8 and a relatively thinner membrane around the indentation surface 1 of the upper and lower layer.

The formation of the passageway in the drug containing layer is controlled by the position of the shallow indentation. The drug is released at rate of quasi-zero order through the passageway by means of the swelling of the osmopolymer when aqueous liquid is absorbed. Hence, drugs with any solubility can be used in the dosage form of the present invention. For example, antiinflammatory, antipyretic, anticonvulsants and/or analgesics such as: indomethacin, diclofenac, sodium diclofenac, codeine, ibuprofen, phenylbutazone, oxyphenbutazone, mepirizol, aspirin, ethenzamide, acetaminophen, aminopyrine, phenacetin, scopolamine butylbromide, morphine, etomidoline, pentazocine, fenoprofen calcium etc.; tuberculostats such as: isoniazid, ethambutol hydrochloride etc.; drugs acting on the cardiovascular system such as: doxazosin, verapamil, isosorbide dinitrate, nitroglycerin, nifedipine, barnidipine hydrochloride, nicardipine hydrochloride, dipyridamole, amrinone, indenolol hydrochloride, hydralazine hydrochloride, methyldopa, furosemide, spirnolactone, guanethidine nitrate, reserpine, amosulalol hydrochloride, etc., antipsychotic agents such as chlorpromazine hydrochloride, amitriptyline hydrochloride, nemonapride, haloperidol, moperone hydrochloride, perphenazine, diazepam, lorazepam, chlordiazepoxide etc., antihistamine such as chlorpheniramine maleate, diphenhydramine hydrochloride etc., vitamins such as thiamine nitrate, tocopherol acetate, cycothiamine, pyridoxal phosphate, cobamamide, ascorbic acid, nicotinamide etc., anti-gout agents such as allopurinol, colchicine, probenecid etc., hypnotic sedatives such as amobarbital, bromovalerylurea, midazolam, chloral hydrate etc., antineoplastic agents such as fluorouracil, carmofur, aclarubicin hydrochloride, cyclophosphamide, thiotepa etc., anticongestants such as phenylpropanolamine, ephedrine etc., antidiabetic agents such as glipizide, acetohexamide, insulin, tolbutamide etc., diuretics such as hydrochlorothiazide, polythiazide, triameterene etc., bronchiectasis agents such as aminophylline, formoterol fumarate, theophylline etc., antitussive agents such as codine phosphate, noscapine, dimemorfan phosphate, dextromethorphan etc., antiarrhythmics such as quinidine nitrate, digitoxin, propafenone hydrochloride, procainamide etc., topical anesthesias such as aminoethylbenzoate, lidocaine, dibucaine hydrochloride etc., antiepileptic agents such as phenytoin, ethosuximide, primidone etc., synthesised adrenocortical steroids such as hydrocortisone, prednioslone, triamcinolone, betamethasone etc., digestive system drugs such as famotidine, ranitidine hydrochloride, cimetidine, sucralfate, sulpiride, teprenone, plaunotol etc., central nervous system drugs such as indeloxazine, tiapride hydrochloride, bifemelin hydrochloride, calciumhopantenate etc., Hyperlipidaemia drugs such as pravastatin sodium etc., and antibiotics such as ampicillin phthalidyl hydrochloride, cefotetan, josamycin, anti-cholinergic agents such as oxybutynin etc. Preferably the solid core of the present invention comprises the following drugs: Verapamil, Glipizide, Doxazosin, Oxybutynin and pharmaceutically acceptable salts thereof. In one preferred embodiment, a solid core contains 0.2˜80 wt % of drug, preferably 1˜35 wt % of drug.

A push layer of an osmotic delivery dosage form based on the present invention comprises osmopolyer. The osmopolymer swells when aqueous liquids are absorbed, for example, poly(hydroxyalkylmethacrylate with a molecular weight of 30,000˜5,000,000, poly(vinylpyrrolidone) with a molecular weight of 10,000˜36,000, anion and cation hydrogels, polyelectrolyte complexes, poly(vinyl alcohol), polyethylene oxide, N-vinyl lactams, Carbopol® acidic carboxy polymer with a molecular weight of 4,000˜4,500,000, Cyanamer® polyacrylamides [symbol] cross-linked water swellable indene-maleic anhydride polymers [symbol] aminopectin copolymer [symbol] Aqua-Keeps® acrylate polymer and polysaccharides etc. The preferable osmopolymer is polyethlene oxide. In one preferred embodiment of the present invention, the push layer comprises an osmopolymer of 5˜90 wt % of solid core, preferably 15˜30 wt %. Also, the drug containing layer comprises an osmopolymer of 5˜90 wt % of solid core, preferably 30˜50 wt %.

An osmotic delivery dosage form based on the present invention can also comprise osmagents if necessary. The osmagent is an organic or inorganic compound which creates osmotic pressure gradient, for example, sodium chloride, potassium chloride, magnesium chloride, magnesium sulfate, mannitol, sorbitol, lactose, glucose, maltose, and potassium phosphate.

Correspondingly, an osmotic delivery dosage form based on the present invention can also comprise effervescent substances if necessary. For example, in one preferred embodiment, the core of the tablet dosage form is a triple-layer tablet (see FIG. 4). The upper layer is an effervescent layer 4 with a shallow indentation. The middle layer is a drug containing layer 2, the lower layer is a push layer 3 comprising an osmopolymer, and the surface is covered by a semipermeable membrane 8. Around the shallow indentation 1, the membrane is relatively thinner. When external aqueous fluids are imbibed through the semipermeable membrane 8 into the dosage form and come into contact with effervescent layer 4, gas is produced and presses the thin membrane around the shallow indentation to form a passageway. The effervescent layer comprises effervescent substances, which is a gas producing compound or salt when it gets in contact with water, such as sodium carbonate, sodium bicarbonate, calcium carbonate etc. In one preferred embodiment, the effervescent layer contains effervescent substances of 1˜20 wt % of the solid core.

Therefore, the present invention also provides an osmotic delivery dosage form, which comprises a) a solid core having a shallow indentation on a surface, which comprises an effervescent layer with a shallow indentation, a drug containing layer containing an active ingredient or pharmaceutically acceptable salts thereof, and a push layer comprising an osmopolymer; and b) a semipermeable membrane having a substantially intact surface enclosing the solid core, wherein the semipermeable membrane completely covers the shallow indentation and is relatively thinner at an indentation position, whereby an in-situ exit passageway is formed in the indentation position when external aqueous fluids are imbibed through the semipermeable membrane into the dosage form by an osmotic pressure gradient. The drug is subsequently released through the passageway.

The present invention also provides a process for forming an in-situ exit passageway of an osmotic delivery dosage form, including: (a) preparing a solid core having a shallow indentation on its surface from osmotic composition containing drugs; (b) covering the solid core completely with a semipermeable membrane and forming a relatively thinner membrane around the indentation surface; and (c) imbibing external aqueous fluids through the semipermeable membrane into the dosage form by an osmotic pressure gradient. A passageway is formed on the indentation surface and the drug is subsequently released through the passageway.

In the present invention, an internal pressure is formed in the dosage form by an osmotic pressure gradient, and subsequently a passageway is generated at the weakest spot of the dosage form. Therefore the lag time for the formation (or the forming time) of the passageway is controlled by the strength of the semipermeable membrane and the pressure gradient. Thereby, it is well-known that by changing the factors which affect the osmotic pressure gradient and strength and permeability of the semipermeable membrane, the internal pressure changing rate and renitency of the weak spot can be regulated. Therefore, by controlling the lag time for the formation of the passageway, the onset time of drug release is controlled. Hence, in the process of the present invention, permeability of the semipermeable membrane, the thickness of the membrane or the osmoagent composition in the solid core, the thickness of the tablets, the depth and area of the shallow indentation etc. can be further adjusted or changed, in order to control the lag time for the formation of the passageway, and the drug is constantly released at quasi-zero order for a certain time. For example, by selecting the polymer, plasticizer, the ratio of the polymer and the plasticizer, or the thickness of the semipermeable membrane, the permeability and toughness of a semipermeable membrane can be changed; adding osmoagent into the formulation of the drug can enhance the osmotic pressure gradient; the methods for modulating the property of a semipermeable membrane and the osmotic pressure gradient are shown in the literature, for example, Journal of Controlled Release 79(2002)7-27.

In the present invention, by controlling the forming time of the passage way, a controlled onset of the drug release can be achieved, which can be applied to chronotherapeutics for some special disease that is more likely to happen in the early morning, such as angina pectoris, apoplexy and asthma.

Hence, the dosage form disclosed in the present invention is also a controlled onset dosage form, including:

a) a solid core having a shallow indentation on a surface of the core, which contains active drug composition and pharmaceutically acceptable salts,

b) a semipermeable membrane having a substantially intact surface enclosing the solid core, wherein the semipermeable membrane completely covers the shallow indentation and is relatively thinner at an indentation position.

When external aqueous fluids are imbibed through the semipermeable membrane into the dosage form by an osmotic pressure gradient, an in-situ exit passageway is formed in the indentation position. The drug is subsequently released through the passageway.

In the present invention, an onset control of the drug release can be achieved by changing the property of a semipermeable membrane or the composition of a solid core. For example, changing the following factors of a dosage form can provide a controlled onset: the thickness of a semipermeable membrane, permeability of a semipermeable membrane, the composition of a solid core, the thickness of a tablet, the shape of a shallow indentation, the area of a shallow indentation or a mixture of the above.

The present invention also provides a controlled onset method, including:

(a) preparing a solid core having a shallow indentation on a surface of the core, which contains active drugs;

(b) enclosing the solid core with a semipermeable membrane having a substantially intact surface, wherein the semipermeable membrane completely covers the shallow indentation and is relatively thinner at an indentation position; and

(c) exposing the dosage form in a liquid environment. When external aqueous fluids are imbibed through the semipermeable membrane into the dosage form by an osmotic pressure gradient, an in-situ exit passageway is formed in the indentation position. The drug is subsequently released through the passageway.

The onset is controlled by the forming time of a passageway, which is described above.

The present invention further provides a method for controlling the time and position of the formation of a passageway, and a method to form a passageway in the human body, the latter including a pharmaceutical composition which comprises: (a) a solid core having a shallow indentation on the surface of the core, which contains active drugs and pharmaceutically acceptable salts; and (b) a semipermeable membrane having a substantially intact surface enclosing the solid core, wherein the semipermeable membrane completely covers the shallow indentation and is relatively thinner at an indentation position. When external aqueous fluids are imbibed through the semipermeable membrane into the dosage form by an osmotic pressure gradient, an in-situ exit passageway is formed in the indentation position. The drug is subsequently released through the passageway.

In the process of the preparation of an osmotic delivery dosage form of the present invention, a medically well-known carrier or excipients can be added to prepare the dosage form. The excipients can be binders, lubricants, disintegrants, fillers etc. They are described in “Handbook of Pharmaceutical Excipients”.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. The present invention is illustrated in the following drawings in which:

FIG. 1 is a display view of a prominence on a mold for preparing a shallow indentation on the surface of a dosage form, wherein

FIG. 1a shows a 3-dimensional view of a mold,

FIG. 1b shows a cross-section view of a mold,

FIG. 1c shows a top view of a mold with a round prominence,

FIG. 1d shows a top view of a mold with a square prominence, and

FIG. 1e shows a top view of a mold with a rhombic prominence.

FIG. 2 shows a preferred construction of an osmotic delivery dosage form in the present invention, wherein

FIG. 2a is a 3-dimensinal view of a double-layer dosage form, and

FIG. 2b shows a cross-section view of a double-layer dosage form.

FIG. 3 is a display view of a preferred construction of a triple-layer tablet, which is an osmotic delivery dosage form in the present invention. The upper and lower layers are both drug containing layers and the middle layer is a push layer.

FIG. 4 is a display view a preferred construction of a triple-layer tablet which is an osmotic delivery dosage form in the present invention. The upper layer is an effervescent layer. The middle layer is a drug layer. The lower layer is a push layer.

FIG. 5 shows a dissolution profile (in 500 ml, pH 7.4 buffer solution) of the tablet made in Example 1 of the present invention according to the method in USP Apparatus II (Paddle).

FIG. 6 shows the profile of dissolution rate vs. time for the tablet made in Example 1 of the present invention.

FIG. 7 shows a dissolution profile (in 500 ml, 0.1N HCl) of the tablet made in Example 2 of the present invention according to the method in USP Apparatus II (Paddle).

FIG. 8 shows a dissolution profile (in 500 ml, pH 7.4 buffer solution) of the tablet made in Example 3 of the present invention according to the method in USP Apparatus II (Paddle).

FIG. 9 shows the profile of dissolution rate vs. time for the tablet made in Example 3 of the present invention.

FIG. 10 shows a dissolution profile (in 900 ml of water) of the tablet made in Example 4 of the present invention according to the method in USP Apparatus II (Paddle).

FIG. 11 shows a dissolution profile (in 900 ml water) of the tablet made in Example 5 of the present invention according to the method in USP Apparatus II (Paddle).

FIG. 12 shows a dissolution profile (in 900 ml water) of the tablet made in Example 6 of the present invention according to the method in USP Apparatus II (Paddle).

FIG. 13 shows a dissolution profile (in 500 ml, pH 7.4 buffer solution) of the tablet made in Example 7 of the present invention according to the method in USP Apparatus II (Paddle).

FIG. 14 shows a dissolution profile (in 500 ml, pH 7.4 buffer solution) of the tablet made in Example 8 of the present invention according to the method in USP Apparatus II (Paddle).

### DESCRIPTION OF THE PREFERRED EMBODIMENTS

Reference will now be made in detail to the present preferred embodiments of the invention, examples of which are illustrated in the accompanying drawings.

### Embodiments

### EXAMPLE 1

The composition of an osmotic delivery dosage form comprising Glipizide:

| Ingredient | WT % | mg |
|---|---|---|
| Glipizide | 7.9 | 20.0 |

Drug Layer

| | | |
|---|---|---|
| Polyox ® N-80 | 43.2 | 110.0 |
| PVP K29-32 | 2.9 | 7.5 |
| sodium chloride (NaCl) | 5.7 | 14.5 |
| magnesium stearate (Mg Stearate) | 0.5 | 1.4 |

Push Layer

| | | |
|---|---|---|
| Polyox ® 303 | 27.8 | 71.0 |
| Sodium chloride | 11.8 | 30.0 |
| Magnesium stearate | 0.2 | 0.5 |

Semipermeable
membrane

| | |
|---|---|
| Cellulose acetate | 75 |
| Hydroxypropylcellulose | 20 |
| Polyethylene glycol-4000 | 5 |
| Acetone:water | 9:1 |

* Polyox ® N-80 is polyethylene oxide (PEO) with molecular weight 200,000. Polyox ® 303 is PEO with molecular weight 700,000. PVP K29-32 is polyvinylpyrrolidone.

a) Preparation of the drug layer: the required amount of Glipizide, sodium chloride, polyethylene oxide Polyox® N-80, polyvinylpyrrolidone, and magnesium stearate, according to the above table, were measured and mixed.

b) Preparation of the push layer: the required amount of sodium chloride, Polyox® 303 and magnesium stearate according to the above table were measured and mixed.

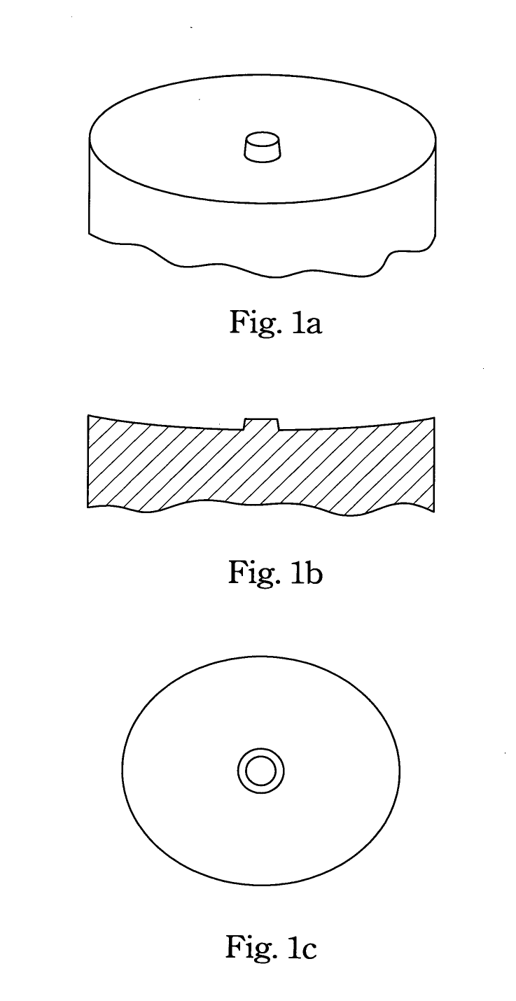

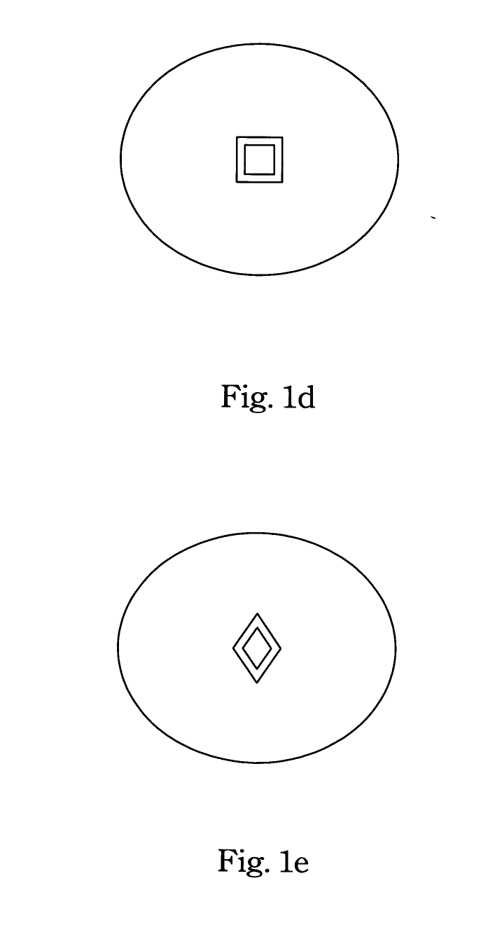

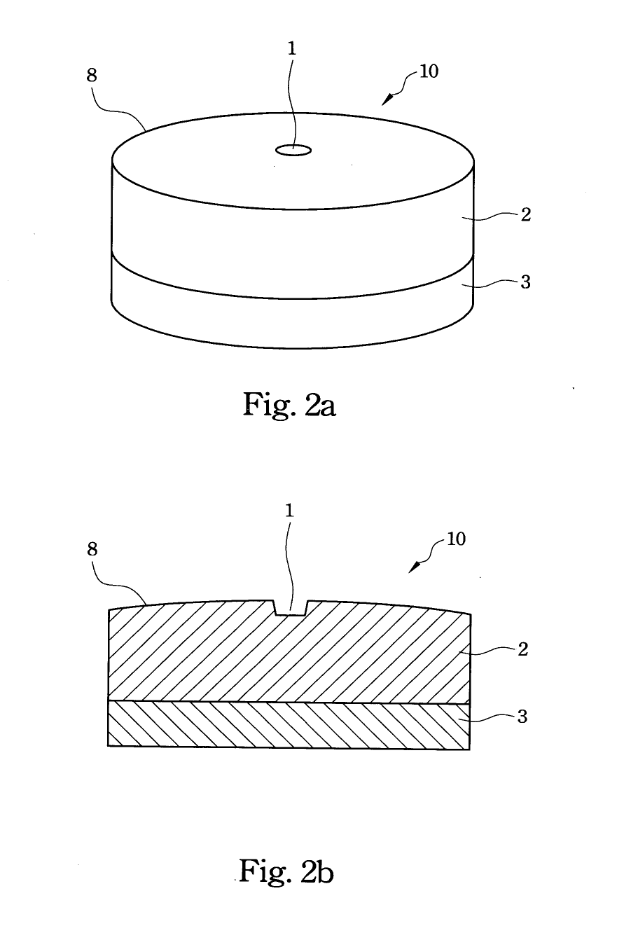

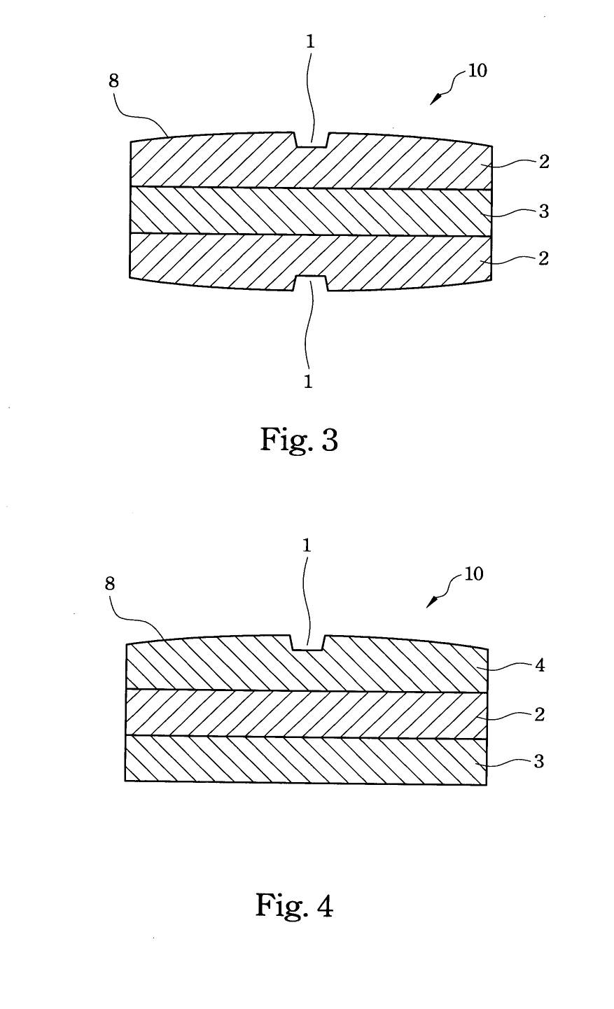

c) Tableting: A round upper punch with a diameter of 9 mm was used. A round prominence with a diameter of 1.5 mm was on the surface of the lower punch (shown in FIG. 1c). Carver Press was used to manufacture tablets. The mixtures of the above drug layer and push layer were compressed into a double-layer non-coated tablet with a shallow indentation on one side of the tablet. The indentation was set on the drug layer with the depth of 200 μm.

d) Controlled Release Coating: The composition of a semipermeable membrane was 75 wt % of cellulose acetate (39.8% of acetyl contained in the molecule), 20 wt % of hydroxypropylcellulose, 5 wt % of polyethylene glycol-4000; 90% of acetone and 10% of water were used as solvent to dissolve the above composition. The membrane was coated around the double-layer tablet with a coating machine. The weight of the coating membrane was about 14% of the non-coated tablet.

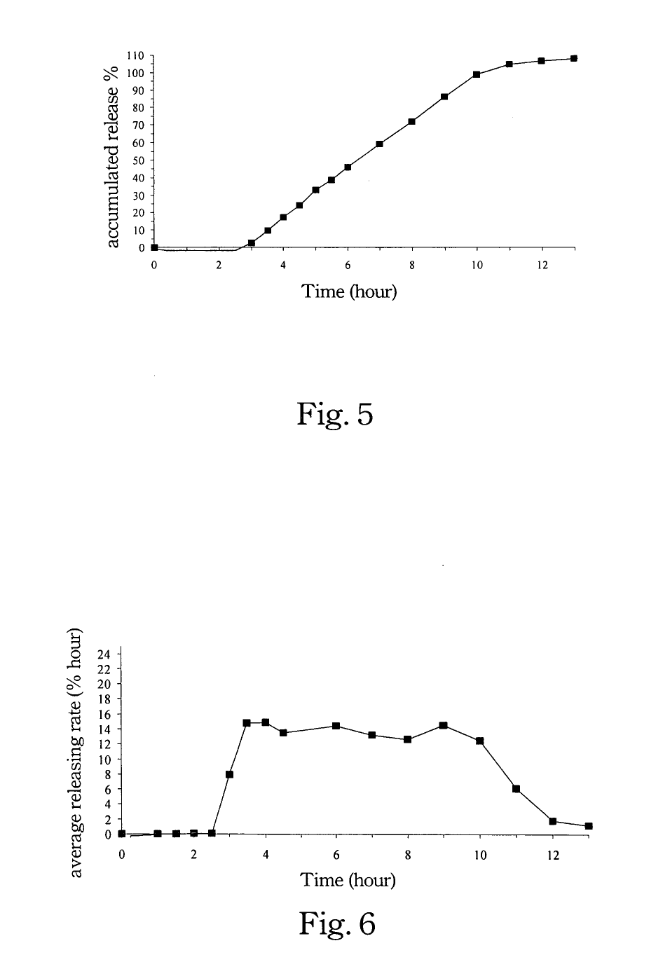

e) According to the United States Pharmacopeia (USP) Apparatus II (Paddle), the dissolution profile (in 500 ml, pH 7.4 buffer solution) for the tablet based on the present formula was shown in FIG. 5. The drug was released after 2.5 hrs of lag time. The profile of dissolution rate to time showed a releasing rate of quasi-zero order (shown in FIG. 6).

### EXAMPLE 2

The composition of an osmotic delivery dosage form comprising Oxybutynin Chloride:

| | | |
|---|---|---|
| Ingredient | WT % | mg |

Drug Layer

| | | |
|---|---|---|
| Oxybutynin Chloride | 6.0 | 15.0 |
| Polyox ® N-80 | 44.0 | 110.0 |
| PVP K29-32 | 3.0 | 7.5 |
| Sodium chloride | 5.8 | 14.5 |
| Magnesium stearate | 0.6 | 1.4 |

Push Layer

| | | |
|---|---|---|
| Polyox ® 303 | 28.4 | 71.0 |
| Sodium chloride | 12.0 | 30.0 |
| Magnesium stearate | 0.2 | 0.5 |

Semipermeable
membrane

| | |
|---|---|
| Cellulose acetate | 75 |
| Hydroxypropylcellulose | 20 |
| Polyethylene glycol-4000 | 5 |
| Acetone:water | 9:1 |

* Polyox ® N-80 was polyethylene oxide (PEO) with molecular weight 200,000. Polyox ® 303 was PEO with molecular weight 700,000.

a) Preparation of the drug layer: the required amount of Oxybutynin Chloride, sodium chloride, polyethylene oxide Polyox® N-80, polyvinylpyrrolidone, and magnesium stearate according to the above table were measured and mixed.

b) Preparation of the push layer: the required amount of sodium chloride (sieve #40 was used to screen), Polyox® 303 and magnesium stearate according to the above table were measured and mixed.

c) Tableting: A round upper punch with a diameter of 9 mm was used. A round prominence with a diameter of 1.5 mm was on the surface of the lower punch (shown in FIG. 1c). Carver Press was used to manufacture tablets. The mixtures of the above drug layer and push layer were compressed into a double-layer non-coated tablet with a shallow indentation on one side of the tablet. The indentation was set on the drug layer with a depth of 200 μm.

d) Controlled Release Coating: The composition of a semipermeable membrane was 75 wt % of cellulose acetate (39.8% of acetyl contained in the molecule), 20 wt % of hydroxypropylcellulose, 5 wt % of polyethylene glycol-4000; 90% of acetone and 10% of water were used as solvent to dissolve the above composition. The membrane was coated around the double-layer tablet with a coating machine. The weight of the membrane was about 10% of the non-coated tablet.

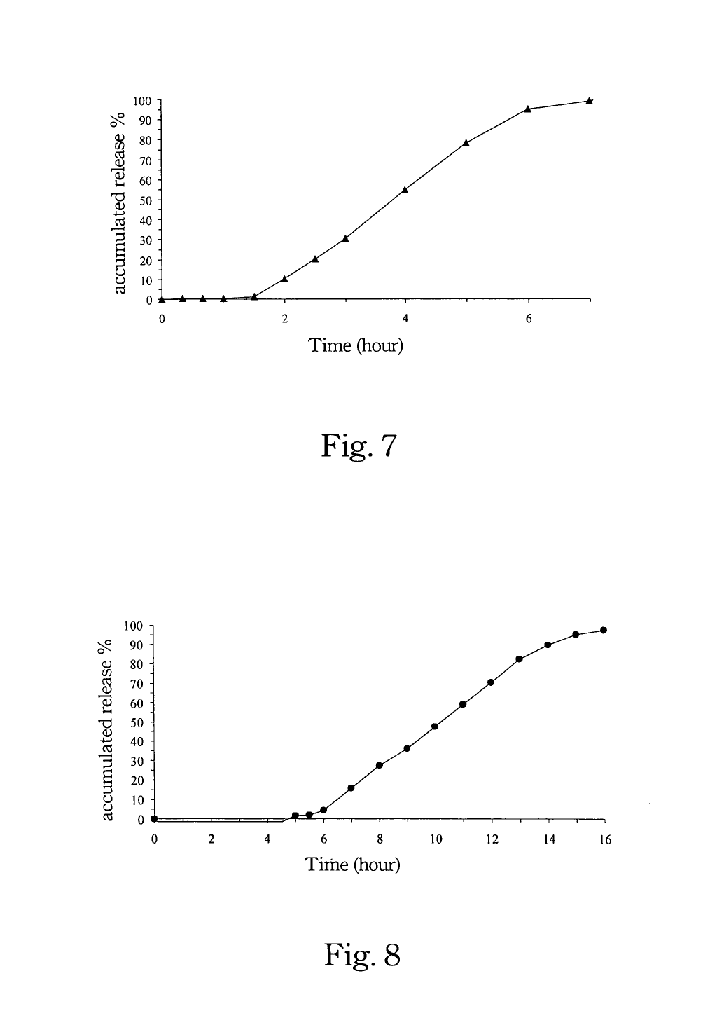

e) According to USP Apparatus II (Paddle), the dissolution profile (in 500 ml, 0.1 N HCl) for the tablet based on the present formula was shown in FIG. 7. The active ingredient was released after 1.5 hrs of lag time. The profile of dissolution rate to time shows a releasing rate of quasi-zero order.

### EXAMPLE 3

An osmotic delivery dosage form comprising Glipizide:

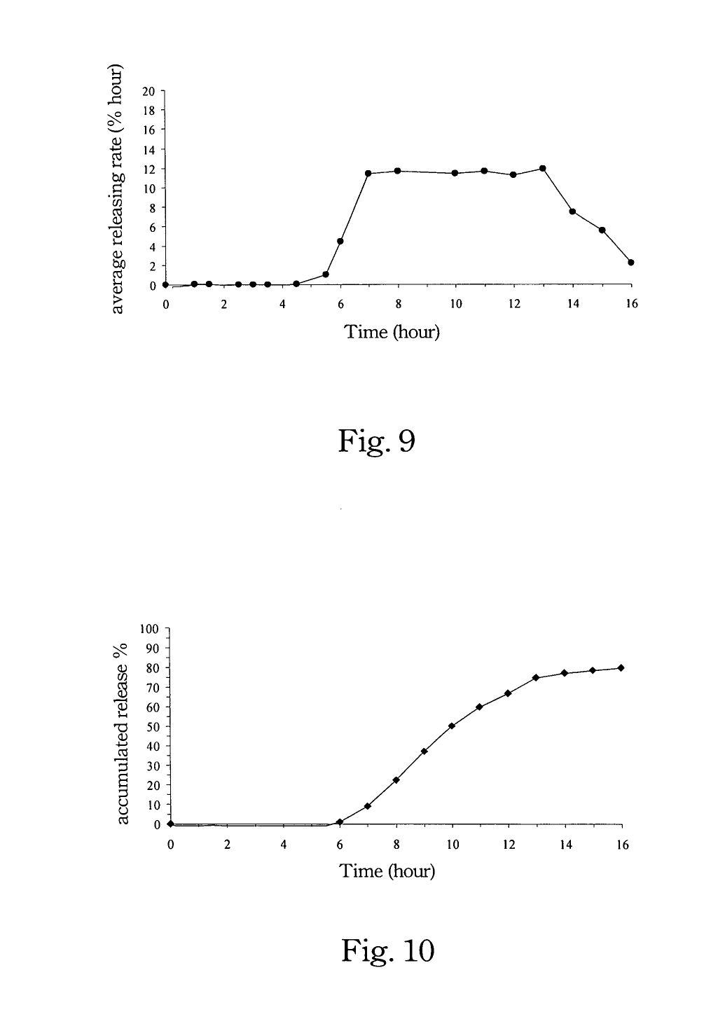

Apart from the change of the following process, the rest process of preparation was identical to example 1. The diameter of the punch was 8.5 mm; the diameter of the prominence on the mold was 1.0 mm; the composition of the semipermeable membrane comprises 93 wt % of cellulose acetate containing 39.8% of acetyl group, 7 wt % of polyethylene glycol-4000; the weight of the membrane was about 14% of the non-coated tablet. The method for measuring the dissolution profile was the same as example 1. The lag time was extended to 5 hrs (shown in FIG. 8). The profile of dissolution rate to time also shows a releasing rate of quasi-zero order (shown in FIG. 9).

The comparison of examples 1 and 3 shows that although the composition of the tablet remains the same, by changing the size of the tablet, the area of the shallow indentation and the composition of the semipermeable membrane, the onset time was extended from 2.5 to 5 hrs.

### EXAMPLE 4

The composition of an osmotic delivery dosage form comprising Verapamil hydrochloride:

| | | |
|---|---|---|
| Ingredient | WT % | Mg |

Drug Layer

| | | |
|---|---|---|
| Verapamil HCl | 22.6 | 55.0 |
| Polyox ® N-80 | 29.4 | 71.6 |
| PVP K29-32 | 3.1 | 7.6 |
| Sodium chloride | 2.7 | 6.5 |
| Magnesium stearate | 0.5 | 1.3 |

Push Layer

| | | |
|---|---|---|
| Polyox ® 303 | 29.2 | 71.0 |
| Sodium chloride | 12.3 | 30.0 |
| Magnesium stearate | 0.2 | 0.5 |

Semipermeable
membrane

| | |
|---|---|
| Cellulose acetate | 93 |
| Polyethylene glycol-4000 | 7 |
| Acetone:water | 9:1 |

* Polyox ® N-80 was polyethylene oxide (PEO) with molecular weight 200,000. Polyox ® 303 was PEO with molecular weight 700,000.

a) Preparation of the drug layer: the required amount of Verapamil HCl, sodium chloride, polyethylene oxide Polyox® N-80, polyvinylpyrrolidone, and magnesium stearate according to the above table were measured and mixed.

b) Preparation of the push layer: the required amount of sodium chloride (sieve #40 was used to screen), Polyox® 303 and magnesium stearate according to the above table were measured and mixed.

c) Tableting: A round punch with a diameter of 8.5 mm was used. A round prominence with a diameter of 1.0 mm was on the surface of the lower punch (shown in FIG. 1c). The mixtures of the above drug layer and push layer were compressed into a double-layer non-coated tablet with a shallow indentation on one side of the tablet. The indentation was set on the drug layer with the depth of 200 μm.

d) Controlled Release Coating: The composition of a semipermeable membrane was 93 wt % of cellulose acetate containing 39.8% of acetyl group, 7 wt % of polyethylene glycol-4000; 90% of acetone and 10% of water were used as solvent to dissolve the above composition. The membrane was coated around the double-layer tablet with a coating machine. The weight of the membrane was about 14% of the non-coated tablet.

According to USP Apparatus II (Paddle), the dissolution profile (in 900 ml water) for the tablet based on the present formula was shown in FIG. 10. The drug was dissolved after 6 hrs of lag time. The profile of dissolution rate to time shows a releasing rate of quasi-zero order.

### EXAMPLE 5

The composition of an osmotic delivery dosage form comprising Doxazosin Mesylate:

| | | |
|---|---|---|
| Ingredient | WT % | mg |

Effervescent
layer

| | | |
|---|---|---|
| NaHCO₃ | 7.0 | 15.0 |
| Maleic Acid | 9.6 | 20.7 |
| Sodium Lauryl Sulfate | 0.8 | 1.8 |

Drug layer

| | | |
|---|---|---|
| Doxazosin Mesylate | 2.3 | 4.85 |
| Polyox ® N-80 | 41.8 | 90.0 |
| PVP K29-32 | 2.8 | 6.0 |
| Sodium chloride | 5.5 | 11.7 |
| Magnesium stearate | 0.5 | 1.0 |

Push layer

| | | |
|---|---|---|
| Polyox ® 303 | 20.9 | 45.0 |
| Sodium chloride | 8.8 | 19.0 |
| Magnesium stearate | 0.1 | 0.3 |

Semipermeable
membrane

| | |
|---|---|
| Cellulose acetate | 65 |
| Hydroxypropylcellulose | 30 |
| Polyethylene glycol-4000 | 5 |
| Acetone:water | 9:1 |

* Polyox ® N-80 was polyethylene oxide (PEO) with molecular weight 200,000. Polyox ® 303 was PEO with molecular weight 700,000.

a) Preparation of the effervescent composition: The required amount of NaHCO₃, Maleic Acid and Sodium Lauryl Sulfate were measured according to the above formula and mixed.

b) Preparation of the drug layer: The required amount of Doxazosin Mesylatel, sodium chloride, polyethylene oxide Polyox® N-80, polyvinylpyrrolidone and magnesium stearate according to the above formula were measured and mixed.

c) Preparation of the push layer: the required amount of sodium chloride (sieve #40 was used to screen), Polyox® 303 and magnesium stearate according to the above table were measured and mixed.

d) Tableting: A round upper punch with a diameter of 8.5 mm was used. A round prominence with a diameter of 1.0 mm (FIG. 1b) was on the surface of the lower punch. The mixtures of the above were compressed into a triple-layer non-coated tablet with a shallow indentation on one side of the tablet. The drug layer was between the push layer and the effervescent layer. The indentation was set on the effervescent layer with the depth of 200 μm.

e) Controlled Release Coating: The composition of a semipermeable membrane was 65 wt % of cellulose acetate (39.8% of acetyl contained in the molecule), 30 wt % of Hydroxypropylcellulose, 5 wt % of polyethylene glycol-4000; 90% of acetone and 10% of water were used as solvent to dissolve the above composition. The membrane was coated around the triple-layer tablet with a coating machine. The weight of the membrane was about 16% of the non-coated tablet.

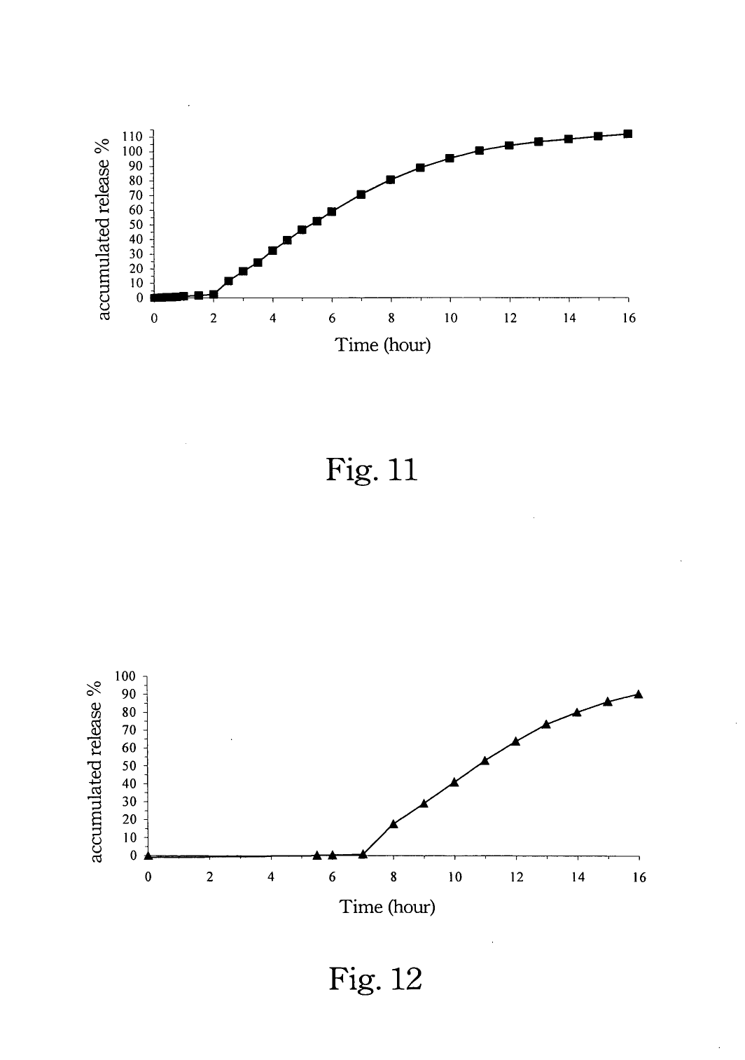

According to the method in USP Apparatus II (Paddle), the dissolution profile (in 900 ml of water) for the tablet based on the present formula was shown in FIG. 11. The drug was released after 2 hrs of lag time. The profile of dissolution rate to time shows a releasing rate of quasi-zero order.

### EXAMPLE 6

The composition of an osmotic delivery dosage form comprising Verapamil HCl:

| | | |
|---|---|---|
| Ingredient | WT % | mg |

Effervescent
layer

| | | |
|---|---|---|
| NaHCO₃ | 5.3 | 15.0 |
| Maleic Acid | 7.4 | 20.7 |
| Sodium Lauryl Sulfate | 0.6 | 1.8 |

Drug layer

| | | |
|---|---|---|
| Verapamil HCl | 19.6 | 55.0 |
| Polyox ® N-80 | 25.5 | 71.6 |
| PVP K29-32 | 2.7 | 7.6 |
| Sodium chloride | 2.3 | 6.5 |
| Magnesium stearate | 0.5 | 1.3 |

Push layer

| | | |
|---|---|---|
| Polyox ® 303 | 25.3 | 71.0 |
| Sodium chloride | 10.7 | 30.0 |
| Magnesium stearate | 0.2 | 0.5 |

Semipermeable
membrane

| | |
|---|---|
| Cellulose acetate | 93 |
| Polyethylene glycol-4000 | 7 |
| Acetone:water | 9:1 |

* Polyox ® N-80 was polyethylene oxide (PEO) with molecular weight 200,000. Polyox ® 303 was PEO with molecular weight 700,000.

a) Preparation of effervescent composition: The required amount of NaHCO₃, Maleic Acid and Sodium Lauryl Sulfate were measured according to the above formula and mixed.

b) Preparation of the drug layer: The required amount of Verapamil HCl, sodium chloride, polyethylene oxide Polyox® N-80, polyvinylpyrrolidone and magnesium stearate according to the above formula were measured and mixed.

c) Preparation of the push layer: the required amount of sodium chloride, Polyox® 303 and magnesium stearate according to the above table were measured and mixed.

d) Tableting: A round upper punch with a diameter of 8.5 mm was used. A round prominence with a diameter of 1.0 mm (FIG. 1b) was on the surface of the lower punch. The mixtures of the above were compressed into a triple-layer non-coated tablet with a shallow indentation on one side of the tablet. The drug layer was between the push layer and the effervescent layer. The indentation was set on the effervescent layer with the depth of 200 μm.

e) Controlled Release Coating: The composition of the semipermeable membrane was 93 wt % of cellulose acetate containing 39.8% of acetyl group, 7 wt % of polyethylene glycol-4000, mixture of 90% of acetone and 10% of water was used as solvent to dissolve the above composition. The membrane was coated around the triple-layer tablet with a coating machine. The weight of the membrane was about 14% of the non-coated tablet.

According to the method in USP Apparatus II (Paddle), the dissolution profile (in 900 ml of water) for the tablet based on the present formula was shown in FIG. 12. The drug was released after 7 hrs of lag time. The profile of dissolution rate to time shows a releasing rate of quasi-zero order.

### EXAMPLE 7

The composition of an osmotic delivery dosage form comprising Glipizide:

| | | |
|---|---|---|
| Ingredient | WT % | mg |

Effervescent
layer

| | | |
|---|---|---|
| NaHCO₃ | 5.1 | 15.0 |
| Maleic Acid | 7.1 | 20.7 |
| Sodium Lauryl Sulfate | 0.6 | 1.8 |

Drug layer

| | | |
|---|---|---|
| Glipizide | 6.8 | 20.0 |
| Polyox ® N-80 | 37.6 | 110.0 |
| PVP K29-32 | 2.6 | 7.5 |
| Sodium chloride | 5.0 | 14.5 |
| Magnesium stearate | 0.5 | 1.4 |

Push layer

| | | |
|---|---|---|
| Polyox ® 303 | 24.3 | 71.0 |
| Sodium chloride | 10.2 | 30.0 |
| Magnesium stearate | 0.2 | 0.5 |

Semipermeable
membrane

| | |
|---|---|
| Cellulose acetate | 75 |
| Hydroxypropylcellulose | 20 |
| Polyethylene glycol-4000 | 5 |
| Acetone:water | 9:1 |

* Polyox ® N-80 was polyethylene oxide (PEO) with molecular weight 200,000. Polyox ® 303 was PEO with molecular weight 700,000.

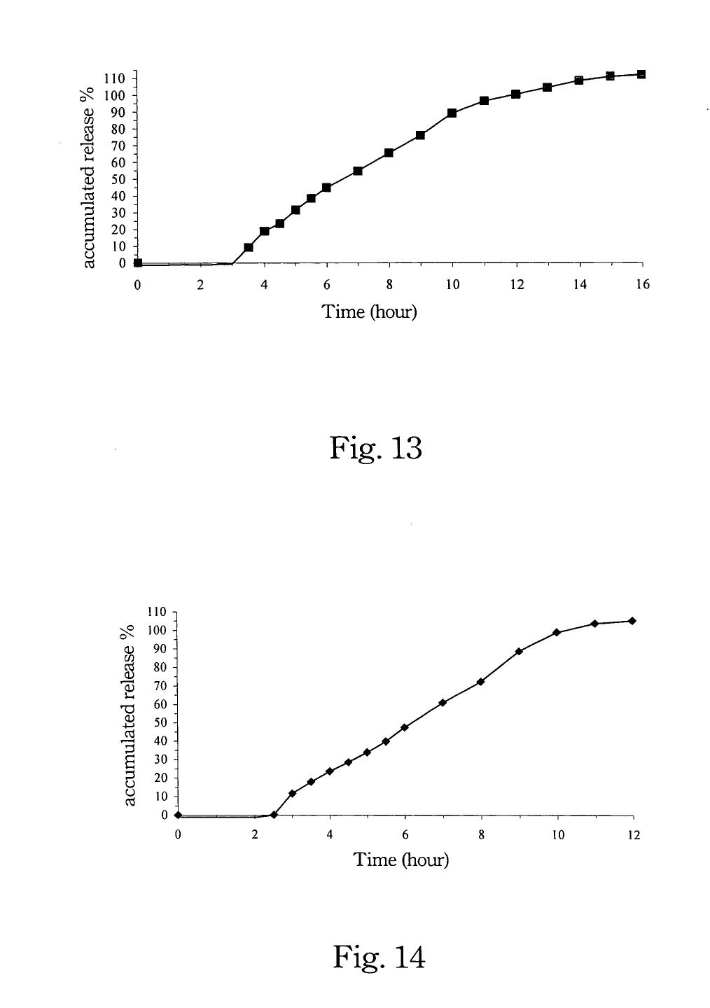

Apart from the change of the following process, the rest process of preparation was identical to example 6: The prominence on the mold was square (FIG. 1d); the composition of the semipermeable membrane comprises 75 wt % of cellulose acetate containing 39.8% of acetyl group, 20 wt % of hydroxypropylcellulose, and 5 wt % of polyethylene glycol-4000; the weight of the membrane was about 10% of the non-coated tablet. The method for measuring the dissolution profile was according to the method in USP Apparatus II (Paddle): The dissolution profile (in 500 ml, pH 7.4 buffer solution) for the tablet based on the present formula was shown in FIG. 13. The drug was released after 3 hrs of lag time. The profile of dissolution rate to time shows a releasing rate of quasi-zero order.

### EXAMPLE 8

The composition of an osmotic delivery dosage form comprising Glipizide:

The prominence on the mold was rhombus (FIG. 1e). The formula and the process of preparation were both identical to example 7. The dissolution profile was shown in FIG. 14. The lag time was 2.5 hrs, and the releasing rate was quasi-zero order.

According to example 1 to example 8 of the present invention, by changing some factors of the method, such as the shape or area of the indentation, composition or ratio of the components of the semipermeable membrane or dosage form or tablet, the onset time can be modulated. Also, the method disclosed in the present invention can be applied to different drugs. The present invention discloses the controlled onset time within 1.5˜7 hrs. Nevertheless, while the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An osmotic delivery dosage form, comprising
(a) a solid core having a shallow indentation on a surface of the solid core and comprising at least a drug or at least a pharmaceutically acceptable salts thereof, and
(b) a semipermeable membrane having a substantially intact surface enclosing the solid core, wherein the semipermeable membrane completely covers the shallow indentation and is relatively thinner at the shallow indentation, when the dosage form is exposed to a liquid environment, a passageway is formed in the indentation position by an osmotic pressure gradient between the solid core and the external liquid environment to osmotically release the drug.

2. The dosage form of claim 1, wherein a depth of the shallow indentation is about 100-300 μm.

3. The dosage form of claim 1, wherein a depth of the shallow indentation is about 150-250 μm.

4. The dosage form of claim 1, wherein the solid core is selected from the group consisting of a single layer tablet, a double-layer tablet, a triple-layer tablet, and a compression-coated tablet.

5. The dosage form of claim 1, further comprising an effervescent substance, an osmagent, an osmopolymer or a mixture thereof.

6. The dosage form of claim 1, wherein the solid core is a double-layer tablet having an upper layer being a drug layer containing the drug and a lower layer being a push layer with an osmopolymer, and the shallow indentation is disposed on a surface of the drug layer.

7. The dosage form of claim 6, wherein the drug in the drug layer is selected from the group consisting of Verapamil, Glipizide, Doxazosin, Oxybutynin and a pharmaceutically acceptable salt thereof.

8. The dosage form of claim 1, wherein the solid core is a triple-layer tablet having an upper layer being an effervescent layer with an effervescent substance, a middle layer being a drug layer containing the drug and a lower layer being a push layer with an osmopolymer, and the shallow indentation is disposed on a surface of the effervescent layer.

9. The dosage form of claim 8, wherein the drug in the drug layer is selected from the group consisting of Verapamil, Glipizide, Doxazosin, Oxybutynin and a pharmaceutically acceptable salt thereof.

10. The dosage form of claim 1, wherein a thickness of the semipermeable membrane is about 100-400 μm.

11. A method to form a passageway of an osmotic delivery dosage form, comprising:
(a) preparing a solid core, having a shallow indentation on a surface of the solid core, from a drug composition that is capable of generating an osmotically effective pressure;
(b) enclosing the solid core with a semipermeable membrane having a substantially intact surface, wherein the semipermeable membrane completely covers the shallow indentation and is relatively thinner at the shallow indentation; and
(c) exposing the dosage form to a liquid environment to form a passageway in the indentation position, as external aqueous fluids in the liquid environment are imbibed into the dosage form by an osmotic pressure gradient, a drug, in the drug composition, is osmotically released through the passageway.

12. The method of claim 11, wherein a depth of the shallow indentation is about 100-300 μm.

13. The method of claim 11, wherein a depth of the shallow indentation is about 150-250 μm.

14. The method of claim 11, wherein the external aqueous fluids are water, gastric and intestinal juice, or imitative gastrointestinal juice.

15. The method of claim 11, wherein the drug is released at a rate of quasi-zero order.

16. The method of claim 11, wherein the drug composition further comprises effervescent substances, osmagents or a mixture thereof.

17. The method of claim 11, further comprising controlling a lag time of forming the passageway in the liquid environment.

18. The method of claim 17, wherein the method for controlling the lag time of forming the passageway in the liquid environment includes adjusting factors selected from the group consisting of a thickness of the semipermeable membrane, permeability of the semipermeable membrane, a composition of the solid core, a thickness of the tablet, a shape of the shallow indentation, an area of the shallow indentation, and a combination thereof.

19. The method of claim 11, wherein the drug composition comprises Verapamil, Glipizide, Doxazosin, Oxybutynin or pharmaceutically acceptable salts thereof.

20. The method of claim 11, wherein the solid core is selected from the group consisting of a single layer tablet, a double-layer tablet, a triple-layer tablet, a multi-layer tablet and a compression-coated tablet.

21. The method of claim 20, wherein the solid core is a double-layer tablet having an upper layer being a drug layer containing the drug and a lower layer being a push layer with an osmopolymer, and the shallow indentation is disposed on a surface of the drug layer.

22. The method of claim 20, wherein the solid core is a triple-layer tablet having an upper layer being an effervescent layer with an effervescent substance, a middle layer being a drug layer containing the drug and a lower layer being a push layer with an osmopolymer, and the shallow indentation is disposed on a surface of the effervescent layer.

23. The method of claim 11, wherein a thickness of the semipermeable membrane is about 100-400 μm.

24. The method of claim 18, wherein the lag time for forming the passageway in the liquid environment is controlled at 1.5˜7 hours.

25. A method of controlling a lag time for formation of a passageway in a dosage form, comprising:
(a) preparing a solid core, having a shallow indentation on a surface of the solid core, from a drug composition being capable of generating an osmotically effective pressure;
(b) enclosing the solid core with a semipermeable membrane having a substantially intact surface, wherein the semipermeable membrane completely covers the shallow indentation and is relatively thinner at the shallow indentation; and
(c) exposing the dosage form to a liquid environment so that a passageway is formed in the indentation position by an osmotic pressure gradient between two sides of the semipermeable membrane to osmotically release the drug.

26. The method of claim 25, wherein the lag time for formation of a passageway is controlled by changing a property of the semipermeable membrane or by changing a composition of the solid core.

27. The method of claim 25, wherein the lag time for formation of a passageway is controlled by changing factors selected from the following group consisting of a thickness of the semipermeable membrane, permeability of the semipermeable membrane, a composition of the solid core, a thickness of the tablet, a shape or area of the shallow indentation and a combination thereof.

28. The method of claim 25, wherein the lag time for formation of a passageway is controlled at 1.5˜7 hours.

29. The method of claim 25, wherein an onset time of a drug is further controlled.

30. The method of claim 29, wherein the method can be applied to chronotherapeutics.
